(19) United States
(12) Patent Application Publication
(10) Pub. No.: US 2026/0294256 A1
(43) Pub. Date: Oct. 1, 2026

(54) Process control system for standardized blood pressure measurement and implementation method

(71) Applicant: JIANGXI CHANGGANG MEDICAL TECHNOLOGY LIMITED COMPANY, Nanchang (CN)

(72) Inventors: Linglin XIA, Nanchang (CN); Yuyuan ZHANG, Nanchang (CN); Aihua YUAN, Nanchang (CN); Renliang GENG, Nanchang (CN); Hai SU, Nanchang (CN)

(21) Appl. No.: 18/688,798

(22) PCT Filed: Apr. 18, 2023
(86) PCT No.: PCT/CN2023/088819

§ 371 Date: Jun. 17, 2026

(30) Foreign Application Priority Data
Nov. 21, 2022 (CN) 202211451732.3

(51) Int. Cl. A61B 5/021; A61B 5/00; G06V 30/148; G06V 30/41

(52) U.S. Cl. CPC A61B 5/02141; A61B 5/7435; G06V 30/148; G06V 30/41; A61B 2560/029

(57) ABSTRACT

Provided are a process control system and an implementation method for standardized blood pressure measurement. The system comprises a blood pressure measurement process control device and a built-in control module. The blood pressure measurement process control device comprises a computer host, a display, a control switch, and an electronic sphygmomanometer. The electronic sphygmomanometer is mounted in a body of the blood pressure measurement process control device by means of a fixing structure. The built-in control module controls the electronic sphygmomanometer to implement a set of blood pressure measurements in a patient, automatically completes the correlation and output of identity information and blood pressure measurement results of the patient, and generates an informationally complete blood pressure measurement report, thus reducing the measurement program error due to human-related causes and improving the credibility of the measurement results and the records while reducing the working intensity of the healthcare providers.

### TECHNICAL FIELD

The present invention relates to the technical field of process control of blood pressure measurement, in particular to a process control system for standardized blood pressure measurement and implementation method.

### BACKGROUND

Modern medicine mostly uses electronic sphygmomanometers to complete the blood pressure measurement of the human body. However, the measurement process of traditional manual control electronic sphygmomanometers is especially arbitrary, and different medical staff will obtain significantly different blood pressure measurement results for the same subject, indicating obvious measurement procedural errors; at the same time, there are also shortcomings in the data collection and management of measurement results. For example, the electronic sphygmomanometer itself only has the function of blood pressure measurement, and does not have the function of entering and binding the subject's identity information. When using it, medical staff usually need to manually record the subject's identity information, which is prone to errors and increases the workload of medical staff. Even an electronic sphygmomanometer with data storage function can only store measurement data in chronological order, the entry of the subject's identity information still needs to be completed manually, and when there are many subjects, it is also easy to cause confusion between the subject's identity information and their blood pressure measurement data, and the blood pressure data of the subject and that of other subjects will appear mutual confusion.

### SUMMARY

In view of the above problems, the present invention proposes a process control system for standardized blood pressure measurement, which realizes a group of standardized blood pressure measurements (bilateral synchronization) for patients by controlling the electronic sphygmomanometer, and automatically completes binding and outputting patient's identity information and blood pressure measurement results, while reducing the work intensity of medical staff, reduce the measurement procedural error caused by human factors, and improve the reliability of measurement results and records.

In order to achieve the above purpose, the present invention adopts the following technical solutions:

A process control system for standardized blood pressure measurement, wherein includes a blood pressure measurement process control device and a built-in control module, the blood pressure measurement process control device includes a computer host, a display screen, a control switch and an electronic sphygmomanometer, and the computer host is used to control the entire measurement process, and the display and output of the results; the display screen is used for human-computer interaction and display of the measurement results; the control switch is used to control the startup and shutdown of the electronic sphygmomanometer; the electronic sphygmomanometer is used to realize blood pressure measurement, and is mounted inside the blood pressure measurement process control device through a fixed structure;

The control module includes an information acquisition module, an image clarity detection module, a data processing module and a blood pressure measurement process control module, wherein the information acquisition module is used to take pictures of the user's identity document information and the measurement results on the display panel of the electronic sphygmomanometer each time, and send the collected image to the image clarity detection module; the image clarity detection module is used to carry out the clarity detection for the collected image, and send the qualified image to the data processing module; the data processing module includes an image preprocessing module, a text detection module, a text recognition module and a regex filtering module, the image preprocessing module is used to crop the document image according to specifications and correct the image taken by the electronic sphygmomanometer display panel, the text detection module and the text recognition module are used to detect and recognize the textual information in the document image and the blood pressure measurement result image, the regex filtering module extracts and fills the key information of the text by specifying the corresponding regex filtering standard, calculates the average and difference values of the blood pressure measurement readings, integrates the user's identity information with the blood pressure measurement results, and then displays it on the display screen.

Further, the control command signal of the control switch is regularly sent out by the built-in program of the signal acquisition module, and the virtual switch is driven by the I/O control card connected through the USB interface to replace the traditional manual start button.

Specifically, the implementation method of the process control system for standardized blood pressure measurement includes the following steps:
Step S1. System environment configuration: placing the blood pressure measurement process control device on a horizontal desktop, connecting the power supply and turning on the power switch of the combined device, and the control system entering a standby state;
Step S2. Identity information acquisition: the user places the identity card or medical insurance card at the designated photo location, and the information acquisition module controls the camera to take photos and samples the user's identity document information, and after the collected photos are qualified by the image clarity detection module, they are sent to the data processing module to generate a list of identity information;
Step S3. Data acquisition and processing: after the identity information is successfully collected, the blood pressure measurement control module controls the electronic sphygmomanometer to complete the user's blood pressure measurement, and the information acquisition module controls the camera to sample the data displayed on the display panel of the electronic sphygmomanometer each time; the data processing module identifies and extracts the sampled data, and further calculates the average and difference values of the blood pressure measurement results to obtain a list of blood pressure measurement results;
Step S4. Measurement report synthesis, printing, and uploading: the data processing module integrates the list of identity information generated in the step S2 with the list of blood pressure measurement results in the step S3, displays it on the display screen, and uploads it to the hospital HIS system or cloud server for storage.

Further, the specific process of the identity information acquisition in the step S2 is as follows:
Step S21. Document image acquisition: the user places the identity card or medical insurance card at the designated photo location, and the information acquisition module controls the camera to take photos, and then the image clarity detection module detects the clarity of the image. If the pixel value of the collected document image is less than the set threshold value of 1080×960, an error message will be returned to prompt the user to re-take and sample a photo; if the pixel value of the collected document image is greater than or equal to the set threshold value of 1080×960, then step S32 will be executed;
Step S22. Document image processing: the image preprocessing module uniformly crops the document images collected in step S31 to an image with an aspect ratio of 3:2 and performs grayscale processing;
Step S23. Document image recognition: using the text detection module and the text recognition module to detect and recognize the textual information in the processed document image, and then obtain the text recognition result of the image after regex filtering;
Step S24. Document information storage and binding: according to the text recognition result obtained in step S33, storing the user's name, gender, and age information to generate a list of identity information, and outputting a blood pressure measurement report after binding with the subsequent list of blood pressure measurement results.

The specific process of the data acquisition and processing is as follows:
Step S31. Display panel image acquisition: the information acquisition module controls the camera to collect photos of the display panel of the electronic sphygmomanometer at the designated location, and after the collected photos are qualified by the image clarity detection module, they are sent to the data processing module;
Step S32. Panel image correction: the image preprocessing module performs further corner detection and affine transformation on the collected image to realize image correction;
Step S33. Character segmentation, recognition and result calculation: the text detection module and the text recognition module complete the independent segmentation of the characters in the corrected image, and use the threading method to perform character recognition on the independently segmented image, and then use the regex filtering module to extract the key information from the recognition text, and calculates the average and difference values of the blood pressure measurement readings.

Further, the specific process of using the text detection module and the text recognition module to detect and recognize the textual information in the processed document image in the step S23 is as follows:
Text detection: using the DBnet network to realize text detection, outputting the text segmentation result of the image through the network based on the segmentation method, using the preset threshold T to convert the segmentation result image into a binary image, inserting the image binarization operation into the network for joint optimization, through more than 4 sampling operations, the network adaptively predicts the threshold of each pixel point in the image, using the ICPR dataset to train the document information model, parsing the text into a list and storing it, then performing recognition after training;
Text recognition: the recognition algorithm is implemented by CRNN+CTC, by extracting features, the text sequence features are extracted based on convolution features, and selecting GRU with lower computational cost; CNN uses Mobilenet to scale to the size of 32×640×32 under the case of fixed aspect ratio of the image, which becomes 1×8×512 after passing through the CNN; setting T=8 for LSTM, and inputting the features into LSTM, in the actual recognition process, taking into account the text repetition, interruption, and different intervals of document information, using CTC to output the results, borrowing the forward-backward algorithm of HMM to predict the blanks in the text by human-specified blank symbols, finally enumerating all possible results, and outputting the recognition text information.

Further, the specific process of using the preset threshold T to convert the segmentation result image into a binary image and the threshold T is obtained by using the OTSU threshold segmentation algorithm is as follows:
\textcircled{1} The size of the blood pressure measurement result image P is width w, height h, the segmentation threshold is T, and T is traversed from 0 to 255;
\textcircled{2} Dividing the image into two parts according to the size of T, and the number of pixels smaller than the threshold T is N₀ and the number of pixels greater than the threshold T is N₁, wherein N₀+N₁=w*h;
\textcircled{3} The proportions of the two divided parts to the entire image P are ω₀ and ω₁, ω₀+ω₁=1; the average gray level are μ₀ and μ₁ separately, wherein μ₀+μ₁=1;
\textcircled{1} The average gray value of the image P is μ, with a variance of ε, ε=ω₀(μ₀−μ)²+ω₁(μ₁−μ)²,
according to the OTSU algorithm, it can be derived:

$$ε = {ω}_{0} {{ω}_{1} ( {μ}_{0} - {μ}_{1} )}^{2} , wherein {ω}_{0} = {N}_{0} / ( w * h ) , {ω}_{1} = {N}_{1} / ( w * h ) ,$$

$$μ = {μ}_{0} {ω}_{0} + {μ}_{1} {ω}_{1} ;$$

By traversing the pixels of the image P, determining the size of the threshold T, that is, the size of the threshold T based on the maximum value of the variance ε.

The beneficial effects of the present invention are:

1. The present invention proposes a process control system for standardized blood pressure measurement, which realizes a group of standardized blood pressure measurements (bilateral synchronization) for patients by controlling the electronic sphygmomanometer, it can reduce the measurement procedural error caused by human factors and obtain more comprehensive information, improve the credibility of the measurement results, and help doctors make more reasonable judgments on the subsequent diagnosis and treatment of hypertensive patients.

2. The present invention proposes an implementation method of a process control system for standardized blood pressure measurement, which can automatically complete binding and outputting patient's identity information and blood pressure measurement results, it can provide convenient and reliable standardized blood pressure measurement process control means and corresponding data management capability, reduce the workload of medical staff.

### BRIEF DESCRIPTION OF THE DRAWINGS

FIG. 1 is a structural block diagram of a standardized double-arm/ankle synchronous blood pressure measurement system of the present invention;

FIG. 2 is a flow chart of a standardized double-arm/ankle synchronous blood pressure measurement method of the present invention;

FIG. 3 is a flow chart of information interaction provided by embodiments of the present invention;

FIG. 4 is a flow chart of image processing provided by embodiments of the present invention.

### DETAILED DESCRIPTION OF THE EMBODIMENTS

In order to make those skilled in the art to better understand the technical solutions in this specification, the technical solutions in the embodiments of this specification will be clearly and completely described below in conjunction with the drawings in the embodiments of this specification. Obviously, the described embodiments are only some of the embodiments of the present application, but not all of them.

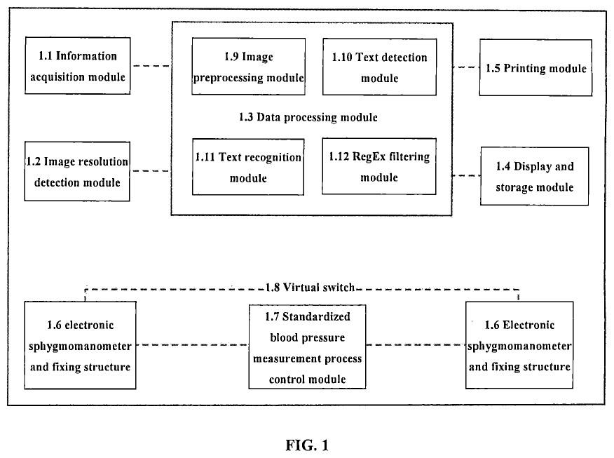

Referring to FIG. 1, a process control system for standardized blood pressure measurement, wherein includes a blood pressure measurement process control device and a built-in control module, the blood pressure measurement process control device includes a computer host, a display screen, a control switch and an electronic sphygmomanometer, and the computer host is used to control the entire measurement process, and the display and output of the results; the display screen is used for human-computer interaction and display of the measurement results; the control switch is used to control the startup, and shutdown of the electronic sphygmomanometer; the electronic sphygmomanometer is used to realize blood pressure measurement, and is mounted inside the blood pressure measurement process control device through a fixed structure;

The control module includes an information acquisition module, an image clarity detection module, a data processing module and a blood pressure measurement process control module, wherein the information acquisition module is used to take pictures of the user's identity document information and the measurement results on the display panel of the electronic sphygmomanometer each time, and send the collected image to the image clarity detection module; the image clarity detection module is used to carry out the clarity detection for the collected image, and send the qualified image to the data processing module; the data processing module includes an image preprocessing module, a text detection module, a text recognition module and a regex filtering module, the image preprocessing module is used to crop the document image according to specifications and correct the image taken by the electronic sphygmomanometer display panel, the text detection module and the text recognition module are used to detect and recognize the textual information in the document image and the blood pressure measurement result image, the regex filtering module extracts and fills the key information of the text by specifying the corresponding regex filtering standard, calculates the average and difference values of the blood pressure measurement readings, integrates the user's identity information with the blood pressure measurement results, and then displays it on the display screen.

Specifically, the control command signal of the control switch is regularly sent out by the built-in program of the signal acquisition module, and the virtual switch is driven by the I/O control card connected through the USB interface to replace the traditional manual start button.

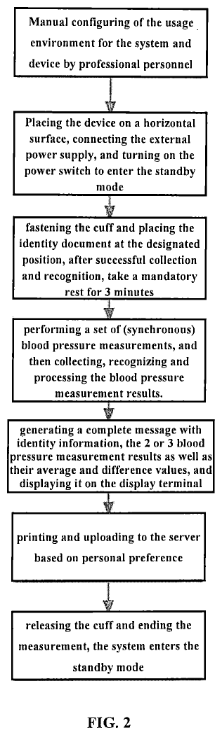

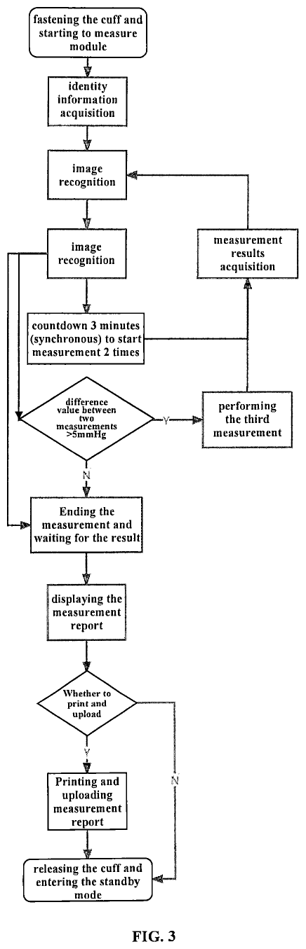

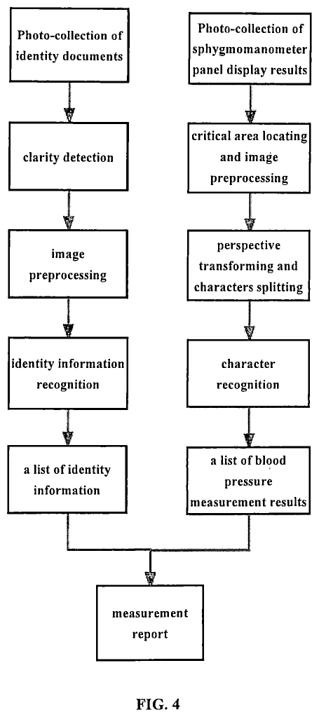

Further, referring to FIG. 2-FIG. 4, the implementation method of the process control system for standardized blood pressure measurement is also provided in this embodiment, including the following steps:
Step S1. System environment configuration: placing the blood pressure measurement process control device on a horizontal desktop, connecting the power supply and turning on the power switch of the combined device, and the control system entering a standby state;
Step S2. Identity information acquisition: the user places the identity card or medical insurance card at the designated photo location, and the information acquisition module controls the camera to take photos and samples the user's identity document information, and after the collected photos are qualified by the image clarity detection module, they are sent to the data processing module to generate a list of identity information;
Step S3. Data acquisition and processing: after the identity information is successfully collected, the blood pressure measurement control module controls the electronic sphygmomanometer to complete the user's blood pressure measurement, and the information acquisition module controls the camera to sample the data displayed on the display panel of the electronic sphygmomanometer each time; the data processing module identifies and extracts the sampled data, and further calculates the average and difference values of the blood pressure measurement results to obtain a list of blood pressure measurement results;
Step S4. Measurement report synthesis, printing, and uploading: the data processing module integrates the list of identity information generated in the step S2 with the list of blood pressure measurement results in the step S3, displays it on the display screen, and uploads it to the hospital HIS system or cloud server for storage.

Further, the specific process of the identity information acquisition in the step S2 is as follows:
Step S21. Document image acquisition: the user places the identity card or medical insurance card at the designated photo location, and the information acquisition module controls the camera to take photos, and then the image clarity detection module detects the clarity of the image. If the pixel value of the collected document image is less than the set threshold value of 1080×960, an error message will be returned to prompt the user to re-take and sample a photo; if the pixel value of the collected document image is greater than or equal to the set threshold value of 1080×960, then step S32 will be executed;
Step S22. Document image processing: the image preprocessing module uniformly crops the document images collected in step S31 to an image with an aspect ratio of 3:2 and performs grayscale processing;
Step S23. Document image recognition: using the text detection module and the text recognition module to detect and recognize the textual information in the processed document image, and then obtain the text recognition result of the image after regex filtering;
Step S24. Document information storage and binding: according to the text recognition result obtained in step S33, storing the user's name, gender, and age information to generate a list of identity information, and outputting a blood pressure measurement report after binding with the subsequent list of blood pressure measurement results.

The specific process of the data acquisition and processing is as follows:
Step S31. Display panel image acquisition: the information acquisition module controls the camera to collect photos of the display panel of the electronic sphygmomanometer at the designated location, and after the collected photos are qualified by the image clarity detection module, they are sent to the data processing module;
Step S32. Panel image correction: the image preprocessing module performs further corner detection and affine transformation on the collected image to realize image correction;
Step S33. Character segmentation, recognition and result calculation: the text detection module and the text recognition module complete the independent segmentation of the characters in the corrected image, and use the threading method to perform character recognition on the independently segmented image, and then use the regex filtering module to extract the key information from the recognition text, and calculates the average and difference values of the blood pressure measurement readings.

Further, the specific process of using the text detection module and the text recognition module described in the step S23 to detect and recognize the textual information in the processed document image is as follows:
Text detection: using the DBnet network to realize text detection, outputting the text segmentation result of the image through the network based on the segmentation method, using the preset threshold T to convert the segmentation result image into a binary image, inserting the image binarization operation into the network for joint optimization, through more than 4 sampling operations, the network adaptively predicts the threshold of each pixel point in the image, using the ICPR dataset to train the document information model, parsing the text into a list and storing it, then performing recognition after training;
Text recognition: the recognition algorithm is implemented by CRNN+CTC, by extracting features, the text sequence features are extracted based on convolution features, and selecting GRU with lower computational cost; CNN uses Mobilenet to scale to the size of 32×640×32 under the case of fixed aspect ratio of the image, which becomes 1×8×512 after passing through the CNN; setting T=8 for LSTM, and inputting the features into LSTM, in the actual recognition process, taking into account the text repetition, interruption, and different intervals of document information, using CTC to output the results, borrowing the forward-backward algorithm of HMM to predict the blanks in the text by human-specified blank symbols, finally enumerating all possible results, and outputting the recognition text information.

Further, the specific process of using the preset threshold T to convert the segmentation result image into a binary image and the threshold T is obtained by using the OTSU threshold segmentation algorithm is as follows:
\textcircled{1} The size of the blood pressure measurement result image P is width w, height h, the segmentation threshold is T, and T is traversed from 0 to 255;
\textcircled{2} Dividing the image into two parts according to the size of T, and the number of pixels smaller than the threshold T is N₀ and the number of pixels greater than the threshold T is N₁, wherein N₀+N₁=w*h;
\textcircled{3} The proportions of the two divided parts to the entire image P are ω₀ and ω₁, ω₀+ω₁=1; the average gray level are μ₀ and μ₁ separately, wherein μ₀+μ₁=1;
\textcircled{4} The average gray value of the image P is μ, with a variance of ε, ε=ω₀(μ₀−μ)²+ω₁(μ₁−μ)²,
according to the OTSU algorithm, it can be derived:

$$ε = {ω}_{0} {{ω}_{1} ( {μ}_{0} - {μ}_{1} )}^{2} , wherein {ω}_{0} = {N}_{0} / ( w * h ) , {ω}_{1} = {N}_{1} / ( w * h ) ,$$

$$μ = {μ}_{0} {ω}_{0} + {μ}_{1} {ω}_{1} ;$$

By traversing the pixels of the image P, determining the size of the threshold T, that is, the size of the threshold T based on the maximum value of the variance ε.

The preferred implementation method of the present invention has been specifically described above, but the invention is not limited to the described embodiments, and those skilled in the art can also make various equivalent variations or substitutions without violating the spirit of the present invention. These equivalent variations or substitutions are all included in the scope defined by the claims of the present application.

What is claimed is:

1. A process control system for standardized blood pressure measurement, comprising a blood pressure measurement process control device having a built-in control module, a display screen, a control switch and an electronic sphygmomanometer, and the control module is configured to control the entire measurement process, and the display and output of the results; the display screen is configured for human-computer interaction and display of the measurement results; the control switch is configured to control the startup and shutdown of the electronic sphygmomanometer; the electronic sphygmomanometer is configured to realize blood pressure measurement, and is mounted inside the blood pressure measurement process control device through a fixed structure;
the control module comprises an information acquisition module, an image clarity detection module, a data processing module and a blood pressure measurement process control module, wherein the information acquisition module is configured to take pictures of the user's identity document information and the information on the display panel of the electronic sphygmomanometer each time, and send the collected image to the image clarity detection module; the image clarity detection module is configured to carry out the clarity detection for the collected image, and send the qualified image to the data processing module; the data processing module comprises an image preprocessing module, a text detection module, a text recognition module and a regex filtering module, the image preprocessing module is configured to crop the document image according to specifications and correct the image taken by the electronic sphygmomanometer display panel, the text detection module and the text recognition module are configured to detect and recognize the textual information in the document image and the blood pressure measurement result image, the regex filtering module extracts and fills the key information of the text by specifying the corresponding regex filtering standard, the data processing module identifies and extracts the sampled data, further calculates the average and difference values of the blood pressure measurement readings, integrates the user's identity information with the blood pressure measurement results, and then displays it on the display screen;
the control command signal of the control switch is regularly sent out by the built-in program of the signal acquisition module, the virtual switch is driven by the I/O control card connected through the USB interface to replace the traditional manual start button, and a set of standardized double-arm synchronous blood pressure measurement of the patient is realized by controlling the electronic sphygmomanometer under the built-in program.

2. (canceled)

3. The system of claim 1, wherein the implementation method of the process control system for standardized blood pressure measurement, comprises:
Step S1: system environment configuration: placing the blood pressure measurement process control device on a horizontal desktop, connecting the power supply and turning on the power switch of the combined device, and the control system entering a standby state;
Step S2: identity information acquisition: the user places the identity card or medical insurance card at the designated photo location, and the information acquisition module controls the camera to take photos and samples the user's identity document information, and after the collected photos are qualified by the image clarity detection module, they are sent to the data processing module to generate a list of identity information;
Step S3: data acquisition and processing: after the identity information is successfully collected, the blood pressure measurement control module controls the electronic sphygmomanometer to complete the user's blood pressure measurement, and the information acquisition module controls the camera to sample the data displayed on the display panel of the electronic sphygmomanometer each time; the data processing module identifies and extracts the sampled data, and further calculates the average and difference values of the blood pressure measurement results to obtain a list of blood pressure measurement results;
Step S4: measurement report synthesis, printing, and uploading: the data processing module integrates the list of identity information generated in the step S2 with the list of blood pressure measurement results in the step S3, displays it on the display screen, and uploads it to the hospital HIS system or cloud server for storage.

4. The system of claim 3, wherein the specific process of the identity information acquisition in the step S2 is as follows:
Step S21: document image acquisition: the user places the identity card or medical insurance card at the designated photo location, and the information acquisition module controls the camera to take photos, and then the image clarity detection module detects the clarity of the image, if the pixel value of the collected document image is less than the set threshold value of 1080×960, an error message will be returned to prompt the user to re-take and sample a photo; if the pixel value of the collected document image is greater than or equal to the set threshold value of 1080×960, then step S32 will be executed;
Step S22: document image processing: the image preprocessing module uniformly crops the document images collected in step S31 to an image with an aspect ratio of 3:2 and performs grayscale processing;
Step S23: document image recognition: using the text detection module and the text recognition module to detect and recognize the textual information in the processed document image, and then obtain the text recognition result of the image after regex filtering;
Step S24: document information storage and binding: according to the text recognition result obtained in step S33, storing the user's name, gender, and age information to generate a list of identity information, and outputting a blood pressure measurement report after binding with the subsequent list of blood pressure measurement results.

5. The system of claim 3, wherein the specific process of the data acquisition and processing in the step S3 is as follows:
Step S31: display panel image acquisition: the information acquisition module controls the camera to collect photos of the display panel of the electronic sphygmomanometer at the designated location, and after the collected photos are qualified by the image clarity detection module, they are sent to the data processing module;
Step S32: panel image correction: the image preprocessing module performs further corner detection and affine transformation on the collected image to realize image correction;
Step S33: character segmentation, recognition and result calculation: the text detection module and the text recognition module complete the independent segmentation of the characters in the corrected image, and use the threading method to perform character recognition on the independently segmented image, and then use the regex filtering module to extract the key information from the recognition text, and calculates the average and difference values of the blood pressure measurement readings.

6. The system of claim 4, wherein the specific process of using the text detection module and the text recognition module to detect and recognize the textual information in the processed document image in the step S23 is as follows:
text detection: using the DBnet network to realize text detection, outputting the text segmentation result of the image through the network based on the segmentation method, using the preset threshold T to convert the segmentation result image into a binary image, inserting the image binarization operation into the network for joint optimization, through more than 4 sampling operations, the network adaptively predicts the threshold of each pixel point in the image, using the ICPR dataset to train the document information model, parsing the text into a list and storing it, then performing recognition after training;
text recognition: the recognition algorithm is implemented by CRNN+CTC, by extracting features, the text sequence features are extracted based on convolution features, and selecting GRU with lower computational cost; CNN uses Mobilenet to scale to the size of 32×640×32 under the case of fixed aspect ratio of the image, which becomes 1×8×512 after passing through the CNN; setting T=8 for LSTM, and inputting the features into LSTM, in the actual recognition process, taking into account the text repetition, interruption, and different intervals of document information, using CTC to output the results, borrowing the forward-backward algorithm of HMM to predict the blanks in the text by human-specified blank symbols, finally enumerating all possible results, and outputting the recognition text information.

7. The system of claim 6, wherein the specific process of using the preset threshold T to convert the segmentation result image into a binary image and the threshold T is obtained by using the OTSU threshold segmentation algorithm is as follows:
the size of the blood pressure measurement result image P is width w, height h, the segmentation threshold is T, and T is traversed from 0 to 255;
dividing the image into two parts according to the size of T, and the number of pixels smaller than the threshold T is N₀ and the number of pixels greater than the threshold T is N₁, wherein N₀+N₁=w*h;
the proportions of the two divided parts to the entire image P are ω₀ and ω₁, ω₀+ω₁=1; the average gray level are μ₀ and μ₁ separately, wherein μ₀+μ₁=1;
the average gray value of the image P is μ, with a variance of ε, ε=ω₀(μ₀−μ)²+ω₁(μ₁−μ)², according to the OTSU algorithm, it can be derived:
$$ε = {ω}_{0} {{ω}_{1} ( {μ}_{0} - {μ}_{1} )}^{2} , wherein {ω}_{0} = {N}_{0} / ( w * h ) , {ω}_{1} = {N}_{1} / ( w * h ) ,$$
$$μ = {μ}_{0} {ω}_{0} + {μ}_{1} {ω}_{1} ;$$
by traversing the pixels of the image P, determining the size of the threshold T, that is, the size of the threshold T based on the maximum value of the variance ε.
